(19) United States
(12) Patent Application Publication
(10) Pub. No.: US 2016/0295848 A1
(43) Pub. Date: Oct. 13, 2016

(54) METHOD FOR TRAPPING RATS, RODENTS, AND THE LIKE

(71) Applicant: ZEEV DORFMAN, Van Nuys, CA (US)

(72) Inventor: ZEEV DORFMAN, Van Nuys, CA (US)

(21) Appl. No.: 14/680,400

(22) Filed: Apr. 7, 2015

(51) Int. Cl. A01M 23/30; A01M 99/00; B65F 1/00; A01N 25/00; A61L 2/18; A01M 23/24; E04B 1/72

(52) U.S. Cl. CPC A01M 23/30; A01M 23/24; A01M 23/245; A01M 99/00; E04B 1/72; A01N 25/004; A61L 2/18; B65F 1/0006; B65F 2240/121

(57) ABSTRACT

A convenient, economical, nontoxic and effective method for the control of rats and the like by administration of one or more snap traps. In the preferred embodiment, rats are exposed to an attractant by means of a disarmed snap trap. In so doing, the rats frequent the disarmed snap trap thereby allowing the rats to feed from the snap trap without being harmed. After a predetermined period of time, preferably several days, the rats will no longer be suspicious of the snap trap because the rats can access the attractant with impunity. The method further requires baiting and arming a snap trap with an attractant after the rats have been allowed to feed from the one or more disarmed snap traps. The rats, unsuspectingly, approach the armed snap trap, which had previously been disarmed, and the spring-loaded trap arm will efficiently trap the rat.

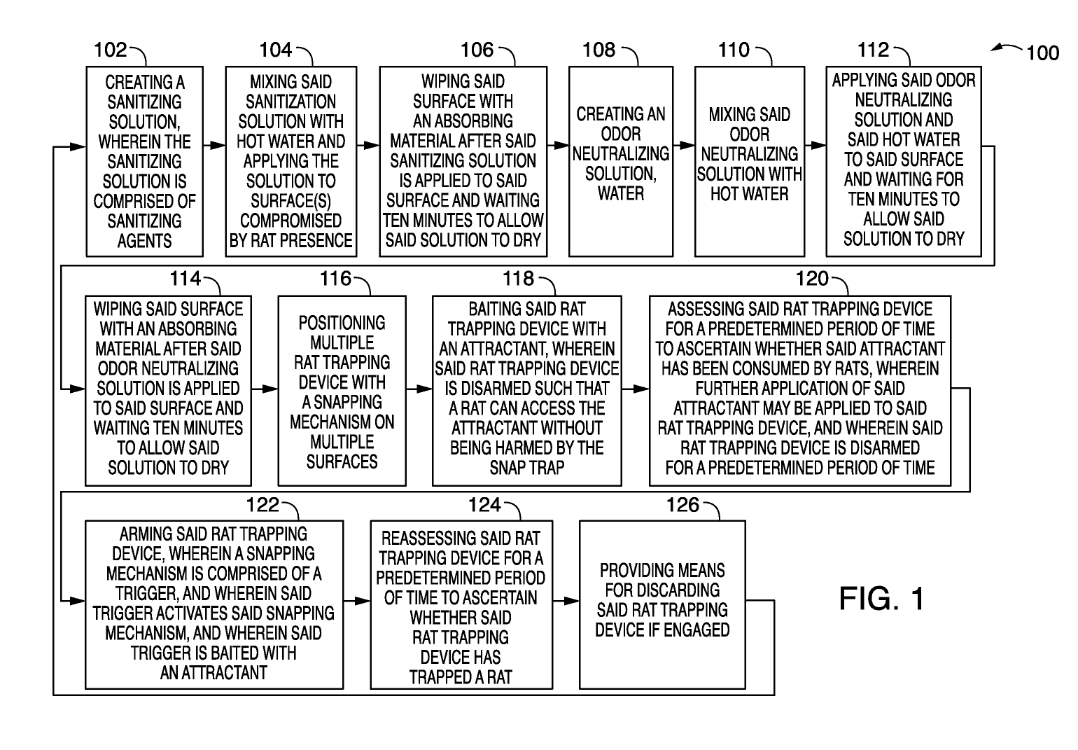

### FIELD OF THE INVENTION

The present invention relates to a method for use in pest control of particularly rats, rodents, and similar animals in a trap.

### BACKGROUND OF THE INVENTION

Rats are responsible for the transmission of many diseases. Their feeding habits are destructive, and their nesting behaviors can compromise the structure of infested buildings. Rat bites and scratches can result in disease, and in some cases, death. Rat urine is responsible for the spread of leptospirosis, which can result in liver and kidney damage where complications include renal and liver failure, as well as cardiovascular problems.

Lymphocytic choriomeningitis (LCMV), a viral infectious disease, is transmitted through the saliva and urine of rats. Some individuals experience long-term effects of lymphocytic choriomeningitis, while others experience only temporary discomfort. One of the most historically dangerous rat-borne diseases is the bubonic plague, also called “Black Plague,” and its variants. Transfer occurs when fleas from the rats bite human beings. From the transmission of bubonic plague to typhus and hantavirus, rat infestations can prove harmful to human health. Their excrement, dander, and hair can cause people to sneeze, experience illness, and experience other allergic reactions. As a result of the aforesaid, pest control is required to shield society from disease-carrying rats, rodents, and the like.

Pest control of rats typically takes place by utilizing poison or by trapping—the former being the most widely used method. The use of poisonous substances is problematic, and the direct drawbacks of the use are well-known. Further, the use of poisonous substances is injurious to humans. For example, humans that unintentionally ingest rat poison may incur serious, irreparable harm to their body. Alternatively, humans that consume livestock, in which said livestock has consumed rat poison may experience negative health effects. Similarly, pets, e.g., dogs, cats, and the like may also suffer serious harm if common rat poisons are consumed. When the rats have been exterminated by consuming the poison, there will typically be leftover residue of the poison called poison deposits such that the surface having contact with the poison will maintain a lingering poisonous residue to the detriment of the environment.

Moreover, one of the greatest problems is considered to be a rapid development of resistance in rats to even the most recent types of poison. According to persons skilled in the pertinent art, this may be an indication that most effective types of rat poisons will be ineffective within a few years. Seen from a humane point of view, the use of the existing poisonous substances may cause the animals to die slowly from internal bleeding, which some would deem unethical and inhumane. Unlike common rat poisons, traps terminate the rat, rodent, or the like in a very expeditious and humane manner.

The term rat will be used to collectively refer to rats, mice, rodents, vermin, and the like within the scope of the present application. The term snap trap collectively refers to conventional spring-loaded rodent traps utilizing a snapping mechanism (e.g., a kill bar, kill jaw, choker, jaw, striking device, trap arm, and the like) designed to be activated through the use of a pressure-sensitive trigger (e.g., a pedal, bait hook, trip pedal, and the like)—the trigger is often baited with an attractant. When the animal nibbles on the attractant that is located on the trigger, the trigger is typically depressed and then activates the snapping mechanism or striking device wherein the striking device springs closed resulting in the instant death of the animal. Subsequently, the bait will be replenished with the expectation that when the rodent attempts to seize the bait, it will cause sufficient depression of the trigger to release the spring-loaded snap arm to strike, trap the rodent.

Popularity of snap traps, which may or may not be rotatably coupled to a platform (or base), is primarily due to their low price and their ability to be both reusable and/or disposable depending on the user's preference. These traps are extremely cost-effective because the traps are reliable, accessible, portable, reusable, disposable, and humane. More importantly, snap traps do not contain any deleterious poisons and/or chemicals, whereas pest control poisons contain extremely harmful toxins. As a result, snap rodent traps are far more popular than competing products in the rodent trap market.

In addition, net traps like glue traps, utilize a method in which the animal is ensnared—whether by an adhesive or a net—and slowly dies due to starvation, lack of oxygen, etc. Further, live capture traps are more expensive than snap traps. Most live capture traps have a box-like structure. To illustrate, when a rat enters the boxes, a mechanized shutter, or the like, is released that closes off the entry point, effectively sealing the animal in the box. Thereafter, the animal is left to die from the elements, starvation, etc.

As noted above, due to the low cost of snap traps, and their wide availability, consumers tend to purchase snap traps more often than any other type of trap with respect to rodent control. In light of the foregoing, a need exists for an effective, novel, and humane method of trapping rats utilizing rodent snap traps.

### SUMMARY OF THE INVENTION

It is an object of the present invention to provide a novel method for trapping rats, which is more effective, efficient, and humane than the known forms.

### BRIEF DESCRIPTION OF THE DRAWINGS

Various objects, features, and attendant advantages of the present invention will become more fully appreciated as the same becomes better understood when considered in conjunction with the accompanying drawing, which is a block diagram showing steps of the method comprising the present invention.

### DETAILED DESCRIPTION

The present invention is an improved method of trapping rats by way of a snap trap, wherein the method can be used with most snap traps available to consumers. The novel method enables consumers to utilize such snap traps in a more effective, efficient, and humane manner—as further described in greater detail below.

As summarized in the Drawing FIGURE, the novel method 100 comprises a step 102 of creating a sanitizing solution comprised of sanitizing agents, disinfecting agents, and the like. A step 104, further requires a mixing of the sanitizing solution with hot water to be applied on all surfaces compromised by rat waste. In a preferred embodiment, the sanitizing solution is nontoxic to humans and pets. Moreover, the sanitizing solution is preferably a disinfectant such that the sanitizing solution will eliminate bacteria and germs associated with rats and their presence. To this end, the sanitizing solution will also remove and mask any traces of human scent that will detract a rat from approaching the surface previously treated with sanitizing solution.

In a step 106, an absorbing material such as a towel, paper towel, cloth, or the like is used to wipe down the surface(s) where sanitizing solution was applied. Thereafter, step 106 further requires allowing ten minutes for the surface(s) and/or the sanitizing solution to dry after the surface(s) has been wiped by the absorbing material.

Subsequently, a step 108 of creating an odor neutralizing solution may be practiced. The neutralizing solution is, preferably, comprised of ingredients which are nontoxic to humans and pets. In addition, the neutralizing solution is further comprised of ingredients which are accessible to consumers and relatively inexpensive. Notably, the neutralizing solution can clean rat waste and neutralize the scent associated with rats. Next, in a step 110, the odor neutralizing solution is mixed with hot water.

Further, in a step 112, the odor neutralizing solution, which was mixed with hot water, is applied to the same affected surface(s) that were treated with the sanitizing solution such that the odor neutralizing solution eliminates the scent markers from which a rat derives, in part, its sense of direction.

In a step 114, much similar to step 106, an absorbing material such as a towel, paper towel, cloth, or the like is used to wipe down the surface(s) where odor neutralizing solution was applied. Thereafter, step 114 further requires allowing ten minutes for the surface to dry after it has been wiped by the aforementioned absorbing material. It is to be understood that the time for the surface to dry will vary.

In a step 116 is further comprised of positioning of a snap trap, or multiple snap traps, on surface(s) wherein rat activity is prevalent and/or observable. In a preferred embodiment, gloves are used to position the traps as rats can detect human scent which will dissuade and deter rats from approaching a trap that emits a human scent.

In a step 118, the snap trap is baited with an attractant, however, the snap trap is disarmed in that a rat can eat the attractant and thereby depress the trigger, however, the spring-loaded snapping mechanism will not strike the rat—because the trap is disarmed. In other words, the snap trap is disengaged thereby enabling the rats to feed on the attractant without harm. It should be appreciated that within the scope of the present application the terms “disengaged” and “disarmed” are interchangeable. What's more, it is important to note that rats are highly suspicious creatures, therefore, it is crucial to build trust with the rats with respect to the snap traps. Rats will not approach suspicious objects. For this reason, snap traps should be disarmed for several days or a predetermined period of time and then positioned where rat activity is suspected. In so doing, the rats are allowed to feed on the attractant; become accustomed to the presence of the trap; and become comfortable with the presence of trap(s).

All too often consumers incorrectly arm a snap trap with an attractant during its first use, however, the majority of rats will be unnerved by the site of an unfamiliar object and be weary upon approaching such an object. Commonly, traps that have been baited incorrectly are ineffective at catching rats because of user error. For example, the user may have used their bare hands to bait and arm the trap—in this case the rats can detect a human scent deterring any rats from approaching the trap regardless of the attractant used—which may be cheese, chocolate, oats, nesting materials, and the like. As such, the snap trap should be baited daily, in a preferred embodiment, with an attractant where the attractant is replenished if necessary. This novel method of delaying the arming of a snap trap with an attractant yields superior results with regards to rat pest control when compared to traditional methods of utilizing conventional rat snap traps.

Moreover, in a step 120, the trap should be assessed daily, or during the predetermined period of time the snap trap was disarmed, to ascertain whether rats are feeding on the attractant and whether the snap trap requires replenishment of an attractant. Preferably, the snap trap is baited with an attractant for several days before being armed. In a preferred embodiment, the snap trap is baited with an ideal attractant such that peanut butter is used. Rats far prefer peanut butter to cheese. This is because peanut butter has a strong smell that attracts rats. Additionally, peanut butter is quite sticky, and it is more difficult for rats to pull off a snap trap. However, the attractant can be comprised of any food a rat would feed on. It is to be appreciated that peanut butter prevents rats from snatching the bait and fleeing before the snap trap catches them. The method of the invention is highly effective in most structures, whereas poisoned bait and similar measures bring, at best, temporary and incomplete relief from the pests.

In addition, the method further comprises a step 122 of arming, the snap trap and baiting the trap with an attractant after the period of time in which it was disarmed. The attractant is comprised of any food that rats feed on, however, it is preferable to use the same attractant throughout the steps of the present method. The step 122 comprises the further step of placing a plurality of snap traps in a structure. In a preferred embodiment, the structure is a dwelling wherein one trap is strategically positioned under the stove; one trap is strategically positioned under the sink; one trap is strategically positioned in the attic; one trap is strategically positioned in the crawl space; and two traps are strategically positioned in areas with obvious signs of rat activity. However, it is to be appreciated that the versatile method of the present invention can also be applied to outdoor rat control.

In a step 124, reassessing the snap trap is further required to determine whether the snap trap has been triggered such that the snap trap has activated the snapping mechanism and must be rearmed. Moreover, if the snap trap requires more attractant, then additional attractant should be applied. In the case that the snap trap has not been triggered and attractant quantity is sufficient, then the snap trap should be revisited after a predetermined period of time. On the other hand, if the snap trap has caught a rat, or the like, then it should be properly discarded properly, as described in step 126.

Finally, a step 126 of discarding a snap trap, that has been triggered, may be practiced in order to properly dispose of a triggered snap trap, a rat, or both utilizing a receptacle. In a preferred embodiment the receptacle is a bag, bin, container, or the like. The user has the choice of discarding both the rat and the trap, however, some users may wish to reuse the traps, and in this case, the user may safely remove the animal from the trap, with gloves, and discard of the animal properly whiling be able to reuse the trap.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A method of trapping rats, comprising:
assessing a structure for evidence of rat activity;
selecting one or more suitable sites for trapping rats;
positioning a snap trap including one or more snap traps at the one or more suitable sites for trapping rats, wherein the snap trap includes a trigger, and wherein the trigger activates a spring-loaded trap arm to trap rats, and wherein said one or more snap traps are disarmed;
applying an attractant on said trigger of said one or more snap traps, wherein the one or more snap traps are disarmed;
allowing said rats to feed on said attractant for a predetermined period of time, wherein said attractant is applied to said trigger of said one or more snap traps, and wherein the one or more said snap traps are disarmed;
determining whether said one or more snap traps require another application of said attractant;
replenishing said attractant to said one or more snap traps, wherein said one or more snap traps are disarmed; and,
arming said one or more snap traps after said predetermined period of time, wherein said one or more snap traps are baited with said attractant.

2. The method of claim 1, wherein the spring-loaded trap arm is comprised of plastic.

3. The method of claim 1, wherein the spring-loaded trap arm is comprised of metal.

4. The method of claim 1, wherein the attractant is selected from the group consisting of chocolate, cheese, peanut butter, and mixtures thereof.

5. The method of claim 1, wherein said structure is a dwelling.

6. The method of claim 1, wherein said structure is a commercial building.

7. The method of claim 1, wherein said one or more suitable sites includes a kitchen.

8. The method of claim 1, wherein said one or more suitable sites includes a crawl space.

9. The method of claim 1, wherein said one or more suitable sites includes an attic.

10. The method of claim 1, wherein said one or more suitable sites includes a locale where rat activity is present.

11. The method of claim 1, further comprising discarding said one or more snap traps with a receptacle.

12. The method of claim 11, wherein said receptacle is a bag.

13. The method of claim 11, wherein said receptacle is a container.

14. The method of claim 1, wherein the absorbing material includes a cloth.

15. The method of claim 1, wherein the absorbing material includes a towel.

16. The method of claim 1, wherein the absorbing material includes a paper towel.

17. The method of claim 1, further comprising applying a sanitizing solution to said one or more suitable sites for trapping rats before an odor neutralizing solution is applied to said one or more suitable sites for trapping rats.

18. The method of claim 17, further comprising applying said odor neutralizing solution to said one or more suitable sites for trapping rats after said sanitizing solution has been applied to said one or more suitable sites.
